(19) United States
(12) Patent Application Publication
(10) Pub. No.: US 2026/0137661 A1
(43) Pub. Date: May 21, 2026

(54) PPAR-ALPHA/GAMMA AGONIST FOR USE IN THE TREATMENT OF LIVER FAILURE

(71) Applicant: Genfit, Loos (FR)

(72) Inventors: Vanessa Legry, Emmerin (FR); Maryse Malysiak, Loos (FR); Simon Debaecker, Pont-A-Marcq (FR); Philippe Poulain, Lorgies (FR)

(21) Appl. No.: 19/121,450

(22) PCT Filed: Nov. 7, 2023
(86) PCT No.: PCT/EP2023/081015

§ 371 Date: Apr. 16, 2025

(30) Foreign Application Priority Data
Nov. 8, 2022 (EP) 22206253.1

(51) Int. Cl. A61K 31/422; A61K 31/255; A61K 31/421; A61P 1/16

(52) U.S. Cl. CPC A61K 31/422; A61K 31/255; A61K 31/421; A61P 1/16

(57) ABSTRACT

The invention relates to compounds for use in the treatment of liver failure.

The invention is in the medical field and relates to compounds for use in the treatment of liver failure.

### BACKGROUND OF THE INVENTION

Liver failure is a severe inability of the liver to perform its normal functions. Manifestations of liver failure herein include acute liver failure (ALF), decompensated cirrhosis, acute cirrhosis decompensation (AD), and acute on chronic liver failure (ACLF).

### Acute Liver Failure (ALF)

The term “ALF” describes a disorder characterized by an acute loss of liver function in the absence of pre-existing chronic liver disease. Acute liver failure is also known as fulminant hepatic failure or fulminant liver failure. ALF has also been referred to as fulminant hepatic failure, acute hepatic necrosis, fulminant hepatic necrosis, and fulminant hepatitis. ALF is a rare and severe consequence of abrupt hepatocyte injury and can evolve over days or weeks to a lethal outcome. A variety of insults to liver cells result in a consistent pattern of rapid-onset elevation of aminotransferases, altered mentation, and disturbed coagulation. The absence of existing liver disease distinguishes ALF from liver failure due to end-stage chronic liver disease (decompensated cirrhosis, acute decompensation and acute-on-chronic liver failure). In ALF, substances that lead to hepatocyte injury cause either direct toxic necrosis, or apoptosis and immune injury, which is a slower process. The time from the onset of symptoms to the onset of hepatic encephalopathy distinguishes the different forms of acute liver failure: a direct, very rapid injury (within hours), referred to as hyperacute liver failure; and a slower, immune-based injury (days to weeks), considered acute or subacute. The term “hepatic encephalopathy”, or HE, as used herein refers to the occurrence of confusion, altered level of consciousness and coma as a result of liver failure. In the advanced stages it is called hepatic coma or coma hepaticum. The five most prevalent causes of ALF in developed countries are paracetamol (acetaminophen) toxicity, ischaemia, drug-induced liver injury, hepatitis B, and autoimmunity, which account for nearly 80% of cases. Hepatitis A, B, and E are the main causes of ALF in developing countries. The remaining causes of ALF comprise fewer than 15% of the total and include heat stroke, pregnancy-associated injury (e.g., acute fatty liver of pregnancy and HELLP [haemolysis, elevated liver enzyme, and low platelet] syndrome), Budd-Chiari syndrome, nonhepatotrophic viral infections such as herpes simplex, and diffusely infiltrating malignancies. Untreated, the prognosis is poor, so timely recognition and management of patients with acute liver failure is crucial. Whenever possible, patients with acute liver failure should be managed in an intensive care unit at a liver transplantation center.

### Decompensated Cirrhosis and Acute Decompensation (AD)

The term “cirrhosis” as used herein, refers to a condition characterized by replacement of liver tissue by fibrosis and regenerative nodules which lead to loss of liver function up to decompensation. Ascites (fluid retention in the abdominal cavity) is the most common complication associated with cirrhosis decompensation. It is associated with a poor quality of life, increased risk of infection and poor long-term outcome. Other potentially life-threatening complications are hepatic encephalopathy and bleeding from esophageal varices. Cirrhosis decompensation has many possible clinical manifestations. These signs and symptoms may be either as a direct result of the failure of liver cells or secondary to the resultant portal hypertension. Effects of portal hypertension include splenomegaly, gastroesophageal varices, and portocollateral circulation as a result of formation of venous collateral veins between portal system and the periumbilical veins as a result of portal hypertension.

Cirrhosis is divided in two clinical categories: compensated and decompensated cirrhosis.

The term “compensated cirrhosis” as used herein, means that the liver is heavily scarred but can still perform many important bodily functions. Patients suffering from compensated cirrhosis experience few or no symptoms and can live without serious clinical complications. Patients at early stages of compensated cirrhosis are characterized by low levels of portal hypertension and lack of esophageal varices. Patients at advanced stages of compensated cirrhosis are characterized by higher levels of portal hypertension and presence of esophageal varices but without ascites and without bleeding.

The term “decompensated cirrhosis” as used herein, means that the liver is extensively scarred and unable to function properly. Patients suffering from decompensated cirrhosis develop a variety of symptoms such as fatigue, loss of appetite, jaundice, weight loss, ascites and/or edema, hepatic encephalopathy and/or bleeding. Patients at early stages of decompensated cirrhosis are characterized by the presence of ascites with or without esophageal varices in a patient that has never bled. Patients at advanced stages of decompensated cirrhosis are characterized by more sever ascites alone or in association with bleeding, bacterial infections and/or hepatic encephalopathy. Complications associated with decompensated cirrhosis such as ascites, edema, bleeding problems, bone mass and bone density loss, hepatomegaly, menstrual irregularities in women and gynecomastia in men, impaired mental status, itching, kidney function failure and muscle wasting can be developed.

The term “acute decompensation” refers to an abrupt deterioration of liver function in patients with advanced chronic liver diseases, compensated cirrhosis or stable decompensated cirrhosis requiring immediate hospitalization. At hospital admission, patients with AD have multiple symptoms including, severe ascites, hepatic encephalopathy, variceal bleeding associated or not with sepsis and/or impaired renal function and/or coagulopathy and/or impaired cardiovascular function and/or impaired respiratory function. AD is a life-threatening condition with an overall mortality rate of 11% at 28-Days.

### Acute on Chronic Liver Failure (ACLF)

ACLF is the most serious hepatic condition observed in patients with known chronic liver disease who have acute decompensation of liver function.

ACLF is an abrupt and life-threatening worsening of clinical conditions in patients with advanced cirrhosis or with cirrhosis due to a chronic liver disease. Three major features characterize this syndrome: it generally occurs in the context of intense systemic inflammation, frequently develops in close temporal relationship with proinflammatory precipitating events (e.g., infections or alcoholic hepatitis), and is associated with single- or multiple-organ failure affecting minimal functioning of vital organs: liver, kidneys, brain, coagulation and/or cardiovascular functions and/or respiratory system. As for sepsis, organ failures are identified with the use of a modified Sequential Organ Failure Assessment score (DOFA score) or the EASL-CLIF Consortium organ failure scoring system), which considers the function of the liver, kidney, and brain, as well as coagulation, circulation, and respiration, allowing stratification of patients in subgroups with different risks of death. Several classifications have been proposed for grading ACLF (APASL, EASL/CLIF, NASCELD). Using the EASL/CLIF, patients were stratified into four prognostic grades according to the number of organ failures at diagnosis (no acute-on-chronic liver failure and acute-on-chronic liver failure grades 1, 2, and 3). Predisposition to ACLF is correlated to the severity (i.e. fibrosis advancement up to cirrhosis) of underlying chronic liver disease. Irrespective of the underlying chronic liver disease, (cholestatic, metabolic liver diseases, chronic viral hepatitis and nonalcoholic steatohepatitis (NASH), alcoholic hepatitis) compensated cirrhosis and stable decompensated cirrhosis are the main conditions associated with development of ACLF. Alcoholic cirrhosis constitutes 50-70% of all underlying liver diseases of ACLF in Western countries, whereas viral hepatitis-related cirrhosis constitutes about 10-30% of all cases.

The severity of underlying disease can be assessed by the Model for End-Stage Liver Disease (MELD) scores.

ACLF requires a precipitating event that occurs in the setting of cirrhosis and/or chronic liver disease and progresses rapidly to multiorgan failure with high mortality. The precipitating events may be reactivation of hepatitis B or superimposed viral hepatitis, alcohol, drugs, ischemic, surgery, sepsis or idiopathic. However, about 40% of patients with ACLF have no precipitating events.

At the onset of liver failure, translocation of bacterial products with or without concomitant translocation of living bacteria from the intestinal lumen plays a pivotal role in development of multiple organ dysfunctions and failures via intense systemic inflammatory response syndrome.

Host response determines the severity of injury. Inflammation and neutrophil dysfunction are of major importance in the pathogenesis of ACLF, and a prominent pro-inflammatory cytokine profile causes the transition from stable decompensated cirrhosis to AD and eventually ACLF. In these patients, an inflammatory response may lead to immune dysregulation, which may predispose to infection that would then further aggravate a pro-inflammatory response resulting in a vicious cycle. Cytokines are believed to play an important role in ACLF. Elevated serum levels of several cytokines, including tumor necrosis factor (TNF)-α, sTNF-αR1, sTNF-αR2, interleukin (IL)-2, IL-2R, IL-4, IL-6, IL-8, IL-10, and interferon-α, have been described in patients with ACLF.

Hyperbilirubinemia is almost invariably present and jaundice is considered an essential criterion of AD and ACLF. Various authors have used different cutoff levels of jaundice, varying from a serum bilirubin of 6-20 mg/dL. Besides jaundice, another hallmark of liver dysfunction is coagulopathy. Coagulation tests are usually abnormal in cirrhotic patients due to impaired synthesis and increased consumption of coagulation factors. Ongoing liver injury culminates in an inexorable downward spiral and death.

The most common organ to fail besides liver is the kidney. Renal failure may be categorized into four types: hepatorenal syndrome, parenchymal disease, hypovolemia-induced and drug-induced renal failure. Bacterial infection (such as spontaneous bacterial peritonitis) is the most common precipitating cause of renal failure in cirrhosis, followed by hypovolemia (secondary to gastrointestinal bleeding, excessive diuretic treatment).

HE is one of the common manifestations of AD and ACLF. HE may be a precipitating factor or a consequence of AD and ACLF. Ammonia is central to the pathogenesis of HE. Indeed, multiple studies have highlighted that hyperammonemia plays a critical role in the development of HE in patients with liver cirrhosis and other liver diseases. Due to liver failure, a large amount of serum ammonia escapes liver metabolism and can reach brain where such high ammonia concentrations are closely related to a high incidence of cerebral edema and herniation. In addition, brain swelling is an important feature of AD and ACLF, similar to the situation in ALF.

One of the hallmark of AD and ACLF is cardiovascular collapse akin to that in patients with ALF. This cardiovascular abnormality is associated with an increased risk of death, particularly in those patients who present renal dysfunction.

Respiratory complications in AD and ACLF can be categorized as acute respiratory failure (e.g., pneumonia) and those that arise as a consequence of cirrhosis (e.g., portopulmonary hypertension and hepatopulmonary syndrome). Patients with cirrhosis are at increased risk of pneumonia.

Patients with AD and ACLF have a statistically higher mortality rate at the same MELD score than patients without ACLF. Regardless of the precipitating event, the final common pathway leading to acute deterioration of liver function and multiorgan failure appears to be an exaggerated activation of systemic inflammation, which is then followed by a period of immune system paralysis. The initial cytokine storm is responsible for profound alterations in macrocirculation, microcirculation, and disruption of normal organ function, resulting in multiorgan failure.

Early interventions to reduce or correct injury are crucial. For patients with more than 3 organ failures, management of ACLF is currently based on the supportive treatment of organ failures, mainly in an intensive care setting. However, the proportion of cases with previous episodes of acute decompensation (development of ascites, encephalopathy, gastrointestinal hemorrhage, bacterial infection) is very frequent in patients with ACLF. Indeed, the appearance of liver failure in a patient with cirrhosis represents a decisive time point in terms of medical management since this condition is frequently associated with rapidly evolving multi-organ dysfunction. The lack of liver detoxification, metabolic and regulatory functions and altered immune response lead to life-threatening complications, such as renal failure, increased susceptibility to infection, hepatic coma and systemic hemodynamic dysfunction. Moreover, only 20% of patients with advanced cirrhosis can be treated with liver transplantation.

There is a need for an adequate treatment of liver failure, in particular of AD, ACLF, ALF and decompensated cirrhosis.

### SUMMARY OF THE INVENTION

The present invention relates to a PPARα/γ agonist selected from aleglitazar, muraglitazar or tesaglitazar, a pharmaceutically acceptable salt thereof, or combinations thereof, for use in a method for the treatment of liver failure in a subject in need thereof.

The present invention also provides the use of a PPARα/γ agonist selected from aleglitazar, muraglitazar or tesaglitazar, a pharmaceutically acceptable salt thereof, or combinations thereof, for the manufacture of a medicament for use in a method for the treatment of liver failure.

The present invention further provides a method for the treatment of liver failure, comprising administering to a subject in need thereof a pharmaceutically effective amount of a PPARα/γ agonist selected from aleglitazar, muraglitazar or tesaglitazar, a pharmaceutically acceptable salt thereof, or combinations thereof.

In a particular embodiment, the compound is aleglitazar or a pharmaceutically acceptable salt thereof.

In a further particular embodiment, the compound is muraglitazar or a pharmaceutically acceptable salt thereof.

In a further particular embodiment, the compound is tesaglitazar or a pharmaceutically acceptable salt thereof.

In a particular embodiment, the PPARα/γ agonist of the invention is for use in the treatment of a liver failure selected from acute decompensation (AD), acute on chronic liver failure (ACLF), acute liver failure (ALF) and decompensated cirrhosis.

In a particular embodiment, the PPARα/γ agonist of the invention is for use in the treatment of AD.

In another particular embodiment, the PPARα/γ of the invention agonist is for use in the treatment of decompensated cirrhosis.

More particularly, the PPARα/γ agonist of the invention is for use in the treatment of ACLF.

In another embodiment, the PPARα/γ agonist of the invention is administered to a subject having AD, decompensated cirrhosis with or without ACLF, or is at risk of AD and ACLF.

In another embodiment, the PPARα/γ agonist of the invention is administered to a subject having decompensated cirrhosis or who is at risk of decompensated cirrhosis or acute decompensation.

In a particular embodiment, the PPARα/γ agonist of the invention is for use in the prevention of decompensated cirrhosis.

In yet another embodiment, the PPARα/γ agonist of the invention is for use in a method for the reversion of decompensated cirrhosis to compensated cirrhosis.

According to another embodiment, the PPARα/γ of the invention agonist is for use in a method for the prevention of liver decompensation in a subject having ACLF.

In another embodiment, the PPARα/γ of the invention agonist is for use in the treatment of ALF.

In another embodiment, the PPARα/γ of the invention agonist is for use in the prevention of kidney failure or in the prevention of hepatic encephalopathy.

According to a particular embodiment, the PPARα/γ agonist of the invention is administered to a subject having ACLF without kidney failure, or to a subject having ACLF with a non-kidney organ failure with kidney dysfunction.

According to another embodiment, the PPARα/γ agonist of the invention is for use in the treatment of sepsis-associated ACLF.

In a particular embodiment, the invention further relates to a method for the treatment of a liver failure selected from acute decompensation (AD), acute on chronic liver failure (ACLF), acute liver failure (ALF) and decompensated cirrhosis AD, comprising administering to a subject in need thereof a pharmaceutically effective amount of the PPARα/γ agonist of the invention.

In another particular embodiment, the invention further relates to a method for the prevention of decompensated cirrhosis, comprising administering to a subject in need thereof a pharmaceutically effective amount of the PPARα/γ agonist of the invention.

In another particular embodiment, the invention further relates to a method for the reversion of decompensated cirrhosis to compensated cirrhosis, comprising administering to a subject in need thereof a pharmaceutically effective amount of the PPARα/γ agonist of the invention.

In another particular embodiment, the invention further relates to a method for the prevention of liver decompensation in a subject having ACLF, comprising administering to said subject a pharmaceutically effective amount of the PPARα/γ agonist of the invention.

In another particular embodiment, the invention further relates to a method for the prevention of kidney failure or in the prevention of hepatic encephalopathy, comprising administering to a subject in need thereof a pharmaceutically effective amount of the PPARα/γ agonist of the invention.

In another particular embodiment, the invention further relates to a method for the treatment of sepsis-associated ACLF, comprising administering to a subject in need thereof a pharmaceutically effective amount of the PPARα/γ agonist of the invention.

In a particular embodiment, the invention further relates to the use of the PPARα/γ agonist of the invention for the manufacture of a medicament for use in a method for the treatment of a liver failure selected from acute decompensation (AD), acute on chronic liver failure (ACLF), acute liver failure (ALF) and decompensated cirrhosis AD.

In another particular embodiment, the invention further relates to the use of the PPARα/γ agonist of the invention for the manufacture of a medicament for use in a method for the prevention of decompensated cirrhosis.

In another particular embodiment, the invention further relates to the use of the PPARα/γ agonist of the invention for the manufacture of a medicament for use in a method for the reversion of decompensated cirrhosis to compensated cirrhosis.

In another particular embodiment, the invention further relates to the use of the PPARα/γ agonist of the invention for the manufacture of a medicament for use in a method for the prevention of liver decompensation in a subject having ACLF.

In another particular embodiment, the invention further relates to the use of the PPARα/γ agonist of the invention for the manufacture of a medicament for use in a method for the prevention of kidney failure or in the prevention of hepatic encephalopathy.

In another particular embodiment, the invention further relates to the use of the PPARα/γ agonist of the invention for the manufacture of a medicament for use in a method for the treatment of sepsis-associated ACLF.

### DESCRIPTION OF THE FIGURES

FIG. 1: Effect of Cpd. 1 (Tesaglitazar) on liver injury and systemic inflammation in a model of acute liver failure. Mice were treated with 1 mg/kg Cpd.1 or vehicle (Veh.) every day for 3 days before LPS/GalN injection. Blood samples were collected 6 hours after LPS/GalN injection for the measurement of serum hepatic markers and cytokines level.

FIG. 1A shows the effect of Cpd. 1 on ASAT after GalN/LPS injection.

FIG. 1B shows the effect of Cpd. 1 on ALAT after GalN/LPS injection.

FIG. 1C shows the effect of Cpd. 1 on total bilirubin after GalN/LPS injection.

FIG. 1D shows the effect of Cpd. 1 on total bile acids after GalN/LPS injection.

FIG. 1E shows the effect of Cpd. 1 on circulating IL6 after GalN/LPS injection. Data are means.

#, ##, ### for p<0.05, p<0.01, p<0.001 for the comparison to the vehicle (Veh.) using 2-tailed Mann-Whitney-test was used to assess statistical significance

FIG. 2: Effect of Cpd. 1 (Tesaglitazar), Cpd.2 (Muraglitazar) and Cpd.3 (Aleglitazar) on LPS activation of THP1 macrophages. After differentiation into macrophages, THP1 cells were treated for 24 h with indicated compound before stimulation with LPS. The cell supernatants were collected 6 hours after LPS to measure MCP1 secretion. The % inhibition of MCP1 secretion was calculated over the mean LPS-vehicle condition (Veh.).

FIG. 2A shows the effect of Cpd. 1 on MCP1 secretion by THP1 differentiated macrophages.

FIG. 2B shows the effect of Cpd.2 on MCP1 secretion by THP1 differentiated macrophages.

FIG. 2C shows the effect of Cpd.3 on MCP1 secretion by THP1 differentiated macrophages. Data are means.

#, ##, ### for p<0.05, p<0.01, p<0.001 for the comparison to untreated condition using a 2-tailed Mann-Whitney-test

$, $$$ for p<0.05, p<0.001 using non-parametric Kruskal-Wallis test to assess statistical significance of compound treatment vs LPS alone

### DETAILED DESCRIPTION OF THE INVENTION

The present invention relates to a PPARα/γ agonist selected from aleglitazar, muraglitazar or tesaglitazar, a pharmaceutically acceptable salt thereof, or combinations thereof, for use in a method for the treatment of a liver failure.

### Definitions

In the context of the present invention, the terms below have the following meanings. Tesaglitazar (Cpd. 1) (also known as AZ 242) is(S)-2-ethoxy-3-(4-(4-((methylsulfonyl)oxy) phenethoxy)phenyl) propanoic acid and corresponds to compound of formula I (CAS No. 251565-85-2):

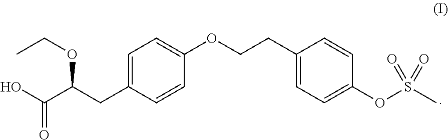 [SMILES: CCO[C@@H](Cc1ccc(OCCc2ccc(OS(C)(=O)=O)cc2)cc1)C(=O)O]

Muraglitazar (Cpd.2) (previously called BMS 298585) is N-[(4-methoxyphenoxy) carbonyl]-N-[[4-[2-(5-methyl-2-phenyl-4-oxazolyl)ethoxy]phenyl]methyl]glycine, also named 2-[(4-methoxyphenoxy) carbonyl-[4-[2-(5-methyl-2-phenyl-1,3-oxazol-4-yl)ethoxy]phenyl]methyl]amino]acetic acid, and corresponds to compound of formula II (CAS No. 331741-94-7):

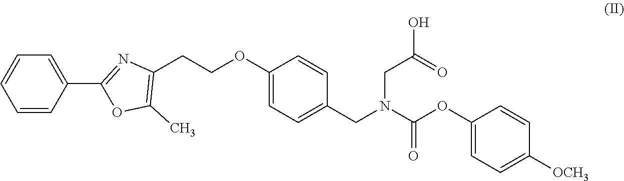 [SMILES: COc1ccc(OC(=O)N(CC(=O)O)Cc2ccc(OCCc3nc(-c4ccccc4)oc3C)cc2)cc1]

Aleglitazar (Cpd.3) (previously called Ro-0728804, R-1439) is (2S)-2-methoxy-3-{4-[2-(5-methyl-2-phenyl-1,3-oxazol-4-yl)ethoxy]-1-benzothiophen-7-yl}propanoic acid and corresponds to compound of formula III (CAS No. 475479-34-6):

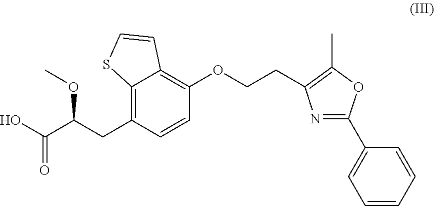 [SMILES: CO[C@@H](Cc1ccc(OCCc2nc(-c3ccccc3)oc2C)c2ccsc12)C(=O)O]

The term “pharmaceutically acceptable salts” includes inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002. The “pharmaceutically acceptable salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine.

As used herein, the terms “treatment”, “treat” or “treating” refer to any act intended to ameliorate the health status of patients such as therapy, prevention, prophylaxis and retardation of a disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult, child, new-born and human at the prenatal stage. However, the term “subject” can also refer to non-human animals, in particular mammals such as dogs, cats, horses, cows, pigs, sheeps and non-human primates, among others.

The expression “substituted by at least” means that the radical is substituted by one or several groups of the list.

In the context of the present invention, the term “about” applied to a numerical value means the value+/−10%. For the sake of clarity, this means that “about 100” refers to values comprised in the 90-110 range. In addition, in the context of the present invention, the term “about X”, wherein X is a numerical value, also discloses specifically the X value, but also the lower and higher value of the range defined as such, more specifically the X value.

### Compounds for Use in the Present Invention

The present invention provides a PPARα/γ agonist selected from aleglitazar, muraglitazar or tesaglitazar, a pharmaceutically acceptable salt thereof, or combinations thereof, for use in a method for the treatment of liver failure.

In a particular embodiment, the PPARα/γ agonist is selected from aleglitazar, muraglitazar, tesaglitazar or combinations thereof. In a particular embodiment, the PPARα/γ agonist is selected from aleglitazar, muraglitazar or tesaglitazar. In a further particular embodiment, the PPARα/γ agonist is tesaglitazar.

In a particular embodiment, the compound for use according to the invention is selected from:
Cpd.1: tesaglitazar;
Cpd.2: muraglitazar; and
Cpd.3: aleglitazar;

In a more particular embodiment, the compound for use according to the invention is Cpd.3: tesaglitazar or a pharmaceutically acceptable salt thereof.

The compound for use according to the invention can be in the form of a pharmaceutically acceptable salt, particularly acid or base salts compatible with pharmaceutical use. Salts of compounds for use according to the invention include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified agonist.

### Liver Failure

In a particular embodiment, the subject is a patient with a liver failure selected in the group consisting of AD, ACLF, ALF and cirrhosis, such as compensated or decompensated cirrhosis. In a particular embodiment, the subject is a patient with a liver failure selected in the group consisting of ACLF, ALF and decompensated cirrhosis.

Alternatively, the subject in need of the treatment is a subject at risk of a liver failure selected from AD, ACLF, ALF and cirrhosis. In a particular embodiment, the subject is at risk of a liver failure selected in the group consisting of AD, ACLF, ALF and decompensated cirrhosis. In particular, the subject may be a patient at risk of AD, ACLF or at risk of decompensated cirrhosis due to a chronic liver disease.

In a particular embodiment, the subject has ALF. In another embodiment, the subject has ALF caused by drug-induced liver injury, paracetamol toxicity, ischaemia, hepatitis A, B or E, autoimmunity, heat stroke, pregnancy-associated injury (e.g., acute fatty liver of pregnancy and HELLP [haemolysis, elevated liver enzyme, and low platelet] syndrome), Budd-Chiari syndrome, nonhepatotrophic viral infections such as herpes simplex and diffusely infiltrating malignancies. In yet another embodiment, the subject has ALF caused by drug-induced liver injury, paracetamol toxicity, ischaemia, hepatitis A, B or E, autoimmunity. In yet another embodiment, the subject has ALF caused by paracetamol toxicity.

In another particular embodiment, the subject is at risk of ALF. In another embodiment, the subject is at risk of ALF caused by drug-induced liver injury, paracetamol toxicity, ischaemia, hepatitis A, B or E, autoimmunity, heat stroke, pregnancy-associated injury (e.g., acute fatty liver of pregnancy and HELLP [haemolysis, elevated liver enzyme, and low platelet] syndrome), Budd-Chiari syndrome, nonhepatotrophic viral infections such as herpes simplex and diffusely infiltrating malignancies. In yet another embodiment, the subject is at risk of ALF caused by drug-induced liver injury, paracetamol toxicity, ischaemia, hepatitis A, B or E, autoimmunity. In yet another embodiment, the subject is at risk of ALF caused by paracetamol toxicity.

In a particular embodiment, the subject has compensated or decompensated cirrhosis, in particular decompensated cirrhosis. In a particular embodiment, the subject has alcoholic cirrhosis, such as alcoholic compensated cirrhosis or alcoholic decompensated cirrhosis, more particularly alcoholic decompensated cirrhosis. In another particular embodiment, the subject has compensated or decompensated cirrhosis consecutive to nonalcoholic fatty liver disease (NAFLD). In another particular embodiment, the subject has decompensated cirrhosis consecutive to nonalcoholic fatty liver disease (NAFLD). In another particular embodiment, the subject has compensated or decompensated cirrhosis consecutive to nonalcoholic steatohepatitis (NASH). In another particular embodiment, the subject has decompensated cirrhosis consecutive to nonalcoholic steatohepatitis (NASH).

In a particular embodiment, the subject is at risk of compensated or decompensated cirrhosis, in particular of decompensated cirrhosis. In a particular embodiment, the subject is at risk of alcoholic cirrhosis, such as of alcoholic compensated cirrhosis or alcoholic decompensated cirrhosis, more particularly of alcoholic decompensated cirrhosis. In another particular embodiment, the subject is at risk of compensated or decompensated cirrhosis consecutive to nonalcoholic fatty liver disease (NAFLD). In another particular embodiment, the subject is at risk of decompensated cirrhosis consecutive to nonalcoholic fatty liver disease (NAFLD). In another particular embodiment, the subject is at risk of compensated or decompensated cirrhosis consecutive to nonalcoholic steatohepatitis (NASH). In another particular embodiment, the subject is at risk of decompensated cirrhosis consecutive to nonalcoholic steatohepatitis (NASH).

In another particular embodiment, the subject has compensated or decompensated cirrhosis and is at risk of AD and ACLF. In another embodiment, the subject has decompensated cirrhosis and is at risk of AD and ACLF.

In another particular embodiment, the subject has ACLF or is at risk of ACLF.

As mentioned above, ACLF is a multiorgan syndrome that generally develops in subjects with cirrhosis, in particular in subjects with decompensated cirrhosis, with at least one organ failure and with high short-term mortality rate. ACLF can develop in patients with chronic liver disease in response to sur-imposed precipitating factors.

In a particular embodiment, the subject suffers from a chronic liver disease with cirrhosis and is at risk of developing ACLF.

The term “chronic liver disease” is used herein to refer to liver diseases associated with a chronic liver injury regardless of the underlying cause. A chronic liver disease may result, for example, from alcohol abuse (alcoholic hepatitis), from viral infectious processes (e.g. viral hepatitis A, B, C, E), autoimmune processes (autoimmune hepatitis), non-alcoholic steatohepatitis (NASH), cancer or chronic exposure to mechanical or chemical injury to the liver. Chemical injury to the liver can be caused by a variety of substances, such as toxins, alcohol, carbon tetrachloride, trichloroethylene, iron or medications.

In a particular embodiment, the subject has a chronic liver disease with cirrhosis. In a particular embodiment, the subject has cirrhosis consecutive to:
alcohol abuse,
viral hepatitis (such as a viral hepatitis resulting from hepatitis A, B, C, D, E, or G virus infection),
use of medication,
metabolic disease,
a biliary disease,
primary biliary cholangitis,
primary sclerosing cholangitis, or
NASH.

The present invention is particularly suitable for the prevention of the recurrence or management of AD and ACLF.

In a particular embodiment, the subject with decompensated cirrhosis, AD or ACLF, shows a high MELD score. The term “MELD score” or “Model for End-Stage Liver Disease” as used herein refers to a scoring system for assessing the severity of liver dysfunction. MELD uses the patient's values for serum bilirubin, serum creatinine and the international ratio for prothrombin time (INR) to predict survival. It is calculated according to the following formula: MELD=3.78 [Ln serum bilirubin (mg/dL)]+11.2 [Ln INR]+9.57 [Ln serum creatinine (mg/dL)]+6.43 wherein, Ln means Napierian logarithm.

Bilirubin is the yellow breakdown product of normal heme catabolism. Bilirubin is excreted in bile and urine. Most bilirubin (70-90%) is derived from hemoglobin degradation and, to a lesser extent, from other hemoproteins. In serum, bilirubin is usually measured as both direct bilirubin and total bilirubin. Direct bilirubin correlates with conjugated bilirubin and it includes both the conjugated bilirubin and bilirubin covalently bound to albumin. Indirect bilirubin correlates to unconjugated bilirubin. The serum bilirubin level can be measured by any suitable method known in the art. Illustrative non-limitative examples of methods for determining serum bilirubin include methods using diazo reagent, methods with DPD, methods with bilirubin oxidase or by means of direct spectrophotometric determination of bilirubin. Briefly, the method for determining the levels of bilirubin in serum with diazo reagents is based on the formation of azobilirubin, which acts as indicator by means of addition of a mixture of sufanilic acid and sodium nitrite. The method based in determining serum bilirubin with DPD is based on the fact that bilirubin reacts with 2,5-dichlorobenzenediazonium salt (DPD) in 0.1 mol/HCl forming azobilirubin with maximal absorbance at 540-560 nm. The staining intensity is proportional to the concentration of bilirubin. Unconjugated bilirubin reacting in the presence of detergent (e.g. Triton TX-100) is determined as total bilirubin whereas only conjugated bilirubin reacts in the absence of detergent. The method for determining the serum level of bilirubin with bilirubin oxidase is based on the reaction catalyzed by the enzyme bilirubin oxidase which oxidizes bilirubin to biliverdin with maximal absorbance at 405-460 nm. The concentration of bilirubin is proportional to the measured absorbance. The concentration of total bilirubin is determined by the addition of sodium dodecyl sulfate (SDS) or sodium cholate which evokes the separation of unconjugated bilirubin from albumin and a reaction of precipitation. The level of serum bilirubin can also be determined by direct spectrophotometric at 454 nm and 540 nm. The measurement at these two wavelengths is used to diminish the hemoglobin interference.

The term “international ratio for prothrombin time”, or “INR” as used herein, refers to a parameter used to determine the clotting tendency of blood. The INR is the ratio of a patient's prothrombin time to a normal (control) sample, raised to the power of the ISI value for the analytical system used. Prothrombin time (PT) measures factors I (fibrinogen), II (prothrombin), V, VII and X and it is used in conjunction with the activated partial tromboplastin time. The prothrombin time is the time it takes plasma to clot after addition of tissue factor. This measures the extrinsic pathway of coagulation. The INR standardizes the results of prothrombin time and is calculated by the following formula: INR=(PTtest/PTnormal)<ISI>

The ISI value of the formula is the International Sensitive Index for any tissue factor and it indicates how a particular batch of tissue factor compares to an international reference tissue factor. The ISI is usually between 1.0 and 2.0.

The value of MELD score correlates strongly with short-term mortality, the lower the value of MELD score the lower the mortality and the higher the value of the MELD score, the higher the mortality. Thus, a patient having low MELD score, for example a MELD lower than 9, has about 1.9% 3-month mortality whereas patients having high MELD score, for example a MELD score of 40 or more, have about 71.3% 3-month mortality.

The term “high MELD score” as used herein, refers to a patient having a MELD score higher than 9, for example, at least 10, at least 15, at least 19, at least 20, at least 25, at least 29, at least 30, at least 35, at least 39, at least 40, at least 45 or more. In a particular embodiment, the present invention is applied to a subject having a MELD score higher than 20.

In another particular embodiment, the patient to be treated shows impairment of kidney function. The term “impairment of kidney function”, also known as “impairment of renal function”, “renal impairment (disorder)”, “renal insufficiency”, “renal impairment” and “renal failure”, as used herein, refers to a medical condition in which the kidneys fail to adequate filter waste products from the blood. Renal failure is mainly determined by a decrease in glomerular filtration rate, the rate which blood is filtered in the glomeruli of the kidney. In renal failure, there may be problems with increased fluid in the body (leading to swelling), increased acid levels, raised levels of potassium, decreased levels of calcium, increased level of phosphate, and in later stages, anemia.

The PPARα/γ agonist as selected above for use according to the invention can be used at any stage of ACLF. In a particular embodiment, the subject has ACLF grade 2 or 3.

In another embodiment, the subject has ACLF without kidney failure. In a particular embodiment, the subject has ACLF with kidney failure. In another particular embodiment, the subject has AD or ACLF with a non-kidney organ failure and kidney dysfunction.

In another embodiment, the subject is at risk of ACLF. In yet another embodiment, the subject has at least one ACLF precipitating event. In another embodiment, the precipitating event is selected from alcoholic hepatitis; bacterial, fungal or viral infection; sepsis, poisoning; visceral bleeding and drug-induced liver insufficiency. In another embodiment, the precipitating event is bacterial infection. In yet another particular embodiment, the PPARα/γ agonist of the invention is for use in a method for the treatment of sepsis-associated AD or ACLF.

In a further embodiment, the PPARα/γ agonist of the invention is for use in a method for treating or preventing hepatic encephalopathy. In a particular embodiment, the PPARα/γ agonist of the invention is for use in a method for treating or preventing hepatic encephalopathy in a subject with compensated or decompensated cirrhosis, in particular with decompensated cirrhosis. In another embodiment, the PPARα/γ agonist of the invention is for use in a method for the treatment of hepatic encephalopathy in a subject with AD or ACLF.

In the context of the present invention, the PPARα/γ agonist of the invention is administered to a subject, in a therapeutically effective amount. A “therapeutically effective amount” refers to an amount of the drug effective to achieve a desired therapeutic result. A therapeutically effective amount of a drug may vary according to factors such as the disease state, age, sex, and weight of the individual, and the ability of drug to elicit a desired response in the individual. A therapeutically effective amount is also one in which any toxic or detrimental effects of agent are outweighed by the therapeutically beneficial effects. The effective dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

The PPARα/γ agonist of the invention can be formulated in a pharmaceutical composition further comprising one or several pharmaceutically acceptable excipients or vehicles (e.g. saline solutions, physiological solutions, isotonic solutions, etc.), compatible with pharmaceutical usage and well-known by one of ordinary skill in the art. These compositions can also further comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. These compositions can be formulated in the form of injectable suspensions, syrups, gels, oils, ointments, pills, tablets, suppositories, powders, gel caps, capsules, aerosols, etc., eventually by means of galenic forms or devices assuring a prolonged and/or slow release. For this kind of formulations, agents such as cellulose, carbonates or starches can advantageously be used.

The PPARα/γ agonist of the invention may be administered by different routes and in different forms. For example, it may be administered via a systemic way, per os, parenterally, by inhalation, by nasal spray, by nasal instillation, or by injection, such as intravenously, by intramuscular route, by subcutaneous route, by transdermal route, by topical route, by intra-arterial route, etc. Of course, the route of administration will be adapted to the form of the drug according to procedures well known by those skilled in the art.

In a particular embodiment, the compound is formulated as a tablet. In another particular embodiment, the compound is administered orally.

The frequency and/or dose relative to the administration can be adapted by one of ordinary skill in the art, in function of the patient, the pathology, the form of administration, etc. Typically, the PPARα/γ agonist of the invention can be administered at a dose comprised between 0.01 mg/day to 4000 mg/day, such as from 50 mg/day to 2000 mg/day, such as from 100 mg/day to 2000 mg/day; and particularly from 100 mg/day to 1000 mg/day. Administration can be performed daily or even several times per day, if necessary. In one embodiment, the compound is administered at least once a day, such as once a day, twice a day, or three times a day. In a particular embodiment, the PPARα/γ agonist is administered once or twice a day. In particular, oral administration may be performed once a day, during a meal, for example during breakfast, lunch or dinner, by taking a tablet comprising the PPARα/γ agonist.

Suitably, the course of treatment with the PPARα/γ agonist of the invention is for at least 1 week, in particular for at least 2, 3, 4, 5, 6, 7, 8, 9, 10, 15, 20 or 24 weeks or more. In a particular embodiment, the course of treatment is for at least 1 month, at least 2 months or at least 3 months. In a particular embodiment, the course of treatment is for at least 1 year, or more depending on the condition of the subject being treated.

In a particular embodiment, the PPARα/γ agonist of the invention (“the drug”), is for use as the sole active ingredient for the treatment disclosed herein.

In yet another embodiment, the drug is for use in a combination therapy.

In a particular embodiment, the drug is for use in combination with therapy against a precipitating event.

In a particular embodiment, the precipitating event is a bacterial, fungal or viral infection. Accordingly, the drug can be combined with an antimicrobial or antiviral agent. The most suitable agent will be selected depending on the organism or virus responsible for the infection, as is well known in the art. In a particular embodiment, the precipitating event is hepatitis B virus reactivation. In that case, the drug can be combined with nucleoside or nucleoside analogues. Illustrative antiviral drugs include, without limitation, tenofovir, tenofovir alafenamide and entecavir. In another particular embodiment, the precipitating event is a bacterial infection, and the drug can be combined to an antibiotic. Antibiotics useful in the treatment of bacterial infection are well known in the art. Illustrative antibiotic families include, without limitation, beta-lactam antibiotics (such as penicillins), tetracyclines, cephalosporins, quinolones, lincomycins, macrolides, sulfonamides, glycopeptides, aminoglycosides and carbapenems. In a particular embodiment, the drug can be combined to an antibiotic of the carbapenem family, such as ertapenem.

In another particular embodiment, the precipitating event is acute variceal hemorrhage. Accordingly, the drug can be combined with a vasoconstrictor such as terlipressin, somatostatin, or analogues such as octreotide or vapreotide, in particular octreotide. Such treatment may accompany endoscopic therapy (preferably endoscopic variceal ligation, performed at diagnostic endoscopy less than 12 hours after admission). Short-term antibiotic prophylaxis, such as with ceftriaxone, can also be implemented.

In another particular embodiment, the precipitating event is alcoholic hepatitis. Accordingly, the drug can be combined with prednisolone, which is indicated for patients with severe alcoholic hepatitis.

In another particular embodiment, the drug is for use in combination with a supportive therapy. In a particular embodiment, the supportive therapy is a cardiovascular support. For example, the drug can be combined with a therapy for acute kidney injury, such as withdrawal of diuretics or volume expansion (with intravenous albumin). The drug may also be combined with a vasoconstrictor, such as terlipressin or norepinephrine, in particular if there is no response to volume expansion. In a particular embodiment, the supportive therapy is a treatment of encephalopathy. For example, the drug can be combined with lactulose. Optionally, lactulose therapy can be further completed with the administration of enemas to clear the bowel. In case the subject has severe hepatic encephalopathy refractory to lactulose, albumin dialysis may be used. In yet another particular embodiment, the drug can be combined with rifaximin. In a further embodiment, the drug can be combined with lactitol. In a particular embodiment, the supportive therapy is an extracorporeal liver support. For example, an extracorporeal liver-assist device that incorporates hepatocytes can be used. In another embodiment, plasma exchange can be conducted in addition to the administration of the drug as provided herein. In yet another embodiment, the extracorporeal liver support is albumin exchange or endotoxin removal.

The following examples serve to illustrate the invention and must not be considered as limiting the scope thereof.

### EXAMPLES

### Chemistry

Chemical names follow IUPAC nomenclature. Cpd. 1 (tesaglitazar), Cpd.2 (muraglitazar) and Cpd.3 (aleglitazar) are well known and are synthetized according to methods known by the man skilled in the art.

Tesaglitazar (Cpd. 1) was synthetized according to method disclosed in WO9962872A1 and it was also purchased from TOCRIS (Ref 3965; batch 1A/263468).

Muraglitazar (Cpd.2) was synthetized following process disclosed in WO2001021602. Aleglitazar (Cpd.3) was synthetized according to method disclosed in WO2002092084A1 and by A. Benardeau et al, Bioorg. Med. Chem. Lett., 2009, vol 19, p 2468-2473.

### Animal Experimentation

Manipulation of animals was conducted carefully in order to reduce stress at the minimum. All the experiments were performed in compliance with the guidelines of French Ministry of Agriculture for experiments with laboratory animals (law 87-848). The study was conducted in compliance with Animal Health Regulation (Council directive No. 2010/63/UE of Sep. 22, 2010 and French decree no. 2013-118 of Feb. 1, 2013 on protection of animals).

### Example 1: Cpd.1 Improves Hepatic Injury and Function and Reduces Systemic Inflammation in a Model of Acute Liver Failure

Low doses of LPS in combination with the hepatotoxic agent D-Galactososamine (GalN) promote specific liver injury in mice and induce the inflammatory cytokines production, thus recapitulating the clinical picture of acute liver injury in human (Pourcet et al., Gastroenterology, 2018, 154 (5), p 1449-1464.e20). Hepatic injury induced by LPS/GalN is therefore a widely used mouse model to evaluate the effect of pharmacological agents on acute liver failure.

### Preclinical Model of Acute Liver Failure

To evaluate the efficacy of the compounds on liver injury and function and the inflammatory response occurring upon acute liver failure, C57BL/6J male mice (8 weeks old, Janvier Labs) received an intraperitoneal injection of 0.025 mg/kg LPS (Escherichia coli 0111: B4, #L2630, Sigma-Aldrich) supplemented with 700 mg/kg D-Galactosamine (GalN, G0500, Sigma-Aldrich).

Cpd.1 (1 mg/kg/day) or vehicle (carboxymethylcellulose 1%, 0.1% Tween 80) was administered by oral gavage during the three days before LPS/GalN injection (n=10-12 per group). Mice were sacrificed 6h after LPS/GalN injection. Blood samples were obtained from retro-orbital sinus puncture on animals slightly asleep with isoflurane (Isoflurin 1000 mg/g, GTIN 03760087152678, Axience) just before sacrifice. A group of mice (n=4) received an intraperitoneal injection to be used as healthy controls.

### Analyses in Mice Serum

Serum aspartate aminotransferase (ASAT) was measured using Randox kit for Daytona Plus automate (AS 8306) according to the manufacturer recommendation. ASAT enzymatically transforms alpha-oxoglutarate and L-aspartate into L-glutamate and oxaloacetate. In the presence of NADH, the generated oxaloacetate is converted by malate dehydrogenase to form L-malate and NAD+. The kinetics of the reaction is studied and allows the concentration of ASAT to be calculated.

Serum alanine aminotransferase (ALAT) was measured using Randox kit for Daytona Plus automate (AL 8304) according to the manufacturer recommendation. ALAT enzymatically transforms alpha-oxoglutarate and L-alanine into L-glutamate and pyruvate. In the presence of NADH, the generated pyruvate is converted by lactate dehydrogenase to form L-lactate and NAD+. The kinetics of the reaction is studied and allows the concentration of ALAT to be calculated.

Serum total bilirubin was measured using Randox kit for Daytona Plus automate (BR 8377) according to the manufacturer recommendation. The bilirubin is oxidized by vanadate at about pH 2.9 to produce biliverdin. In the presence of detergent and vanadate, both conjugate and unconjugated bilirubin are oxidized. This oxidation reaction causes a decrease in the optical density of the yellow color, which is specific to bilirubin. The decrease in optical density at 450/546 nm is proportional to the total bilirubin concentration in the sample.

Serum total bile acids were measured using Randox kit for Daytona Plus automate (BI 3863) according to the manufacturer recommendation. In the presence of Thio-NAD, the enzyme 3-α-hydroxysteroid dehydrogenase (3-α-HSD) converts bile acids to 3-keto steroids and Thio-NADH. In the presence of excess NADH, the enzyme cycling occurs efficiently and the rate of formation of Thio-NADH is determined by measuring specific change of absorbance at 405 nm.

The concentration of serum interleukin-6 (IL6) was determined using a multiplex sandwich ELISA system (Mouse Magnetic Luminex #LSXAMSM-06, Biotechne) according to the manufacturer instructions. Briefly, serum samples were added onto magnetic particles pre-coated with cytokines-specific antibodies. After washing, IL6 was detected through the addition of biotinylated antibodies. Finally, streptavidin conjugated with phycoerythrin were added and analysis were carried out with the Luminex 200 analyzer. The signal strength of phycoerythrin is proportional to the concentration of the specific cytokine.

### Results

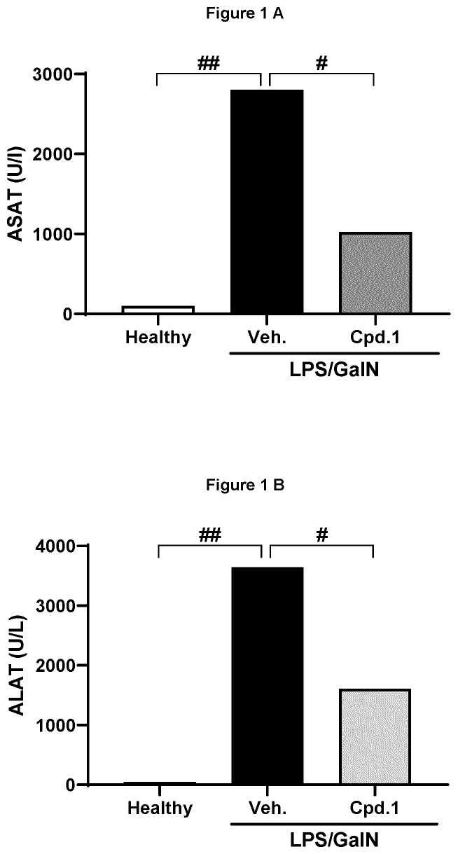

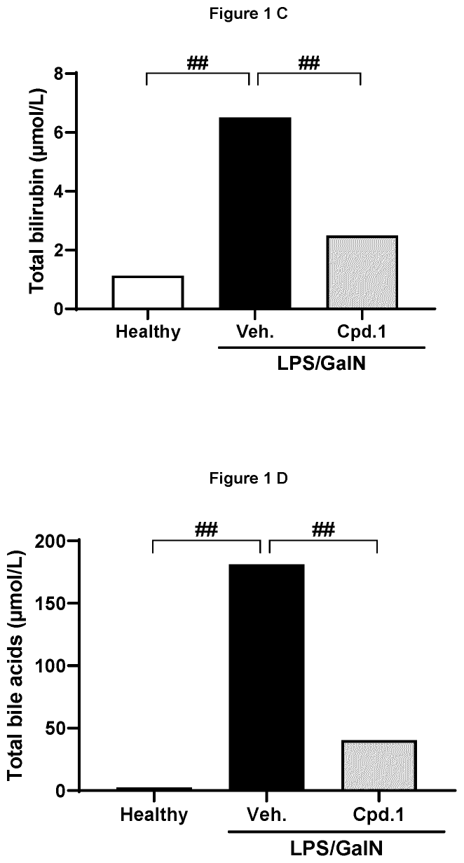

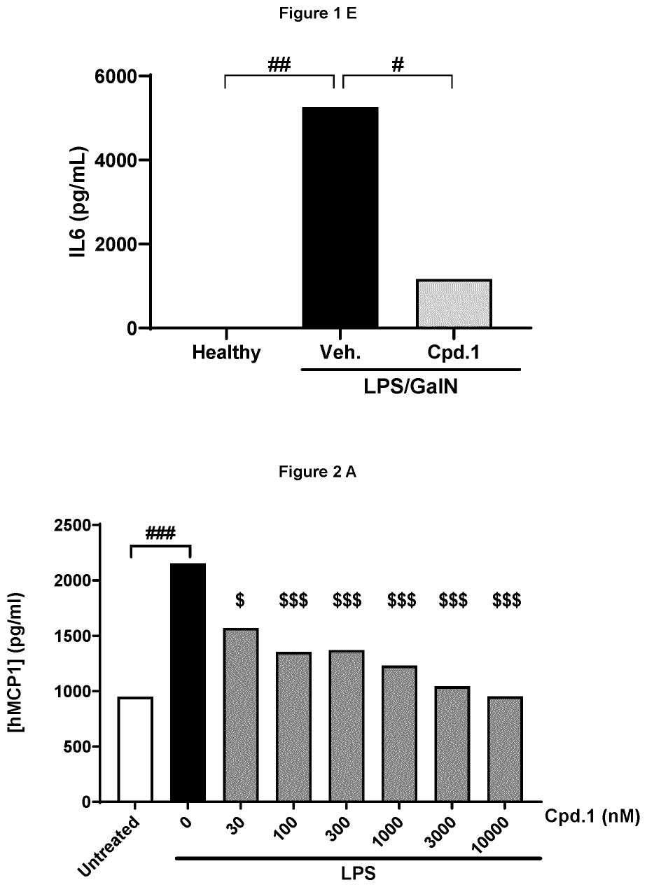

As expected in this model, mice injected with LPS/GalN had serious liver injury as demonstrated by very high levels of ASAT (>2000 U/L) and ALAT (>3000 U/L) (FIG. 1A-B). Cpd. 1 greatly alleviated liver injury by decreasing ASAT and ALAT by 66% (p=0.01) and 57% (p=0.03), respectively (FIG. 1A-B). Hepatic functions, such as metabolism of bilirubin and bile acids, were also strongly altered in this model while Cpd. 1 greatly improved these markers, as demonstrated by a reduction of 73% of total bilirubin (p=0.003) and 79% of total bile acids (p=0.004) (FIG. 1 C-D). Interestingly, these hepatoprotective effects were associated with anti-inflammatory effects as attested by a significant reduction of the pro-inflammatory cytokine IL6 (−78%, p=0.01) (FIG. 1 E).

These results demonstrate that Cpd. 1 exerts hepatoprotective and anti-inflammatory effects thereby alleviating the liver damages and alterations of hepatic functions in acute liver failure.

### Example 2: The Compounds According to the Invention Inhibit Macrophage Activation

Human monocytic cell line THP-1 (Sigma) were used to test the efficacy of the compounds to inhibit the activation of immune cells. THP1 monocytes were cultured in RPMI 1640 with L-glutamine medium (#10-040-CV, Corning) supplemented with 10% fetal bovine serum (FBS, #10270, Gibco), 1% penicillin/streptomycin (#15140, Gibco) and 25 mM Hepes (H0887, Sigma) in a 5% CO2 incubator at 37° C.

Cpd.1 was purchased from TOCRIS (Ref 3965; batch 1A/263468).

In order to test the efficacy of the compounds on macrophage activation, 2.5×10⁴ THP-1 cells were cultured in a 384-well plate and treated with 100 ng/mL PMA (#P8139, Sigma) for 24h to induce differentiation into macrophages. Then, medium was removed and FBS-deprived medium containing the compounds was added for 24h. Finally, THP1 macrophages were stimulated with 100 ng/mL LPS (Klebsiella pneumoniae, #L4268, Sigma-Aldrich) for 6h.

Monocyte chemoattractant protein 1 (MCP1) was measured in cell supernatants by Homogeneous Time Resolved Fluorescence (HTRF) (62HMCP1PEG, Cisbio). Fluorescence was measured with Infinite 500 (#30019337, Tecan) to determine MCP1 concentration.

### Results

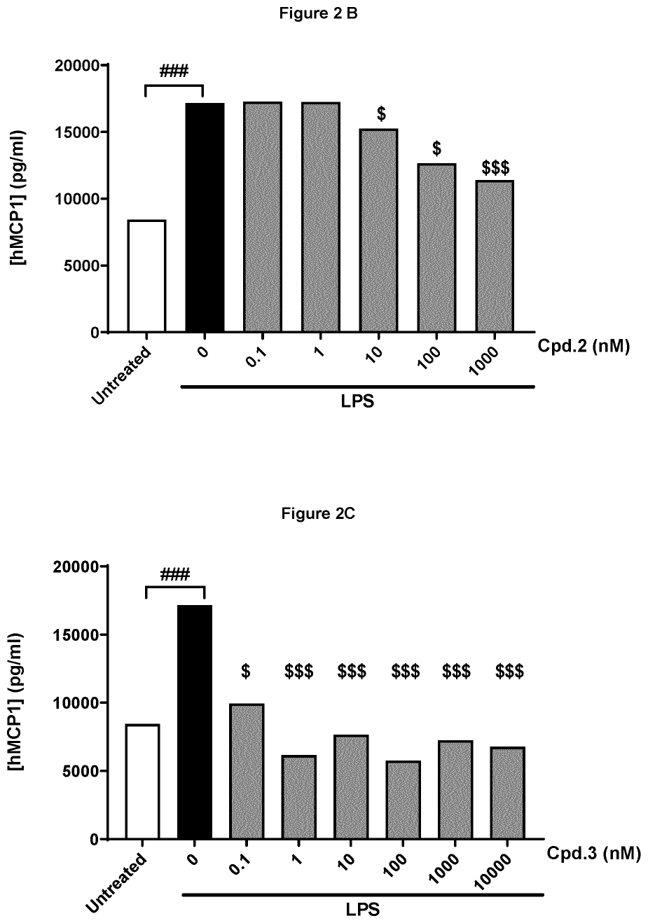

Treatment of macrophages with LPS led to a 2-fold increase of MCP1 levels (FIGS. 2A, B and C). As shown in FIG. 2A, Cpd. 1 reduced the level of LPS-induced MCP1 secretion in a dose-dependent manner, reaching 100% inhibition at 10 μM (p<0.001). Treatment of THP1 macrophages with Cpd.2 and Cpd.3 also showed a dose-dependent decrease of MCP1 secretion, reaching 66% inhibition for Cpd. 2 at 1 UM and 130% inhibition for Cpd.3 at 0.1 μM (FIG. 2 B-C).

These results show the potency of the compounds of the invention to counteract macrophage activation, thereby protecting damages to the tissues induced by over activation of the immune system.

### Example 3: Compounds According to the Invention Protect Hepatocytes from Apoptosis

Whether in healthy patients or in patients with a fibrotic liver due to an underlying chronic liver disease, hepatocyte death is a hallmark of liver failure and can be induced by a variety of stress factors (alcohol, drug, cytokine storm, etc).

In order to evaluate the effect of the compounds to protect hepatocytes from cell death, apoptosis was induced by staurosporine in the human hepatoblastoma-derived HepG2 cell line (ECACC, #85011430, Sigma-Aldrich). HepG2 was cultured in high-glucose DMEM medium (#41965, Gibco, France) supplemented with 10% of fetal bovine serum (FBS, #10270, Gibco), 1% penicillin/streptomycin (#15140, Gibco), 1% sodium pyruvate (#11360, Gibco) and 1% MEM non-essential amino acids (#11140, Gibco) in a 5% CO2 incubator at 37° C.

To evaluate caspase 3/7 activity, which is a surrogate marker of apoptosis, 1.5×10⁴ cells were plated in a 384-well plate (#781080, Greiner, France). After cell adherence (8 hours), cells were serum starved for 16h in the presence of compounds (dose ranging from 0.3 to 10 UM) or vehicle. Thereafter, cells were treated with 10 μM staurosporin (#569397, Sigma-Aldrich, Germany) supplemented with compound for additional 4 hours before cell lysis and caspase activity measurement.

Caspase 3/7 activity was measured using Caspase Glow™ 3/7 assay (#G8093, Promega, USA). Luminescence was measured using a Spark microplate reader (#30086376, Tecan, USA). The amount of luminescence (RLU) directly correlates with caspase 3/7 activity.

### Results

Incubation of HepG2 cells with staurosporine induced apoptosis, as shown by a dramatic increase in caspase 3/7 activity by 5-fold. Interestingly, the 3 compounds according to the invention significantly inhibit Caspase 3/7 activity in a dose dependent manner: Cpd. 1 reaches 12% inhibition (p<0.001) at the dose of 10 μM, Cpd.2 reaches 24% inhibition (p<0.001) at the dose of 3 μM, Cpd.3 reaches 17% inhibition (p<0.001) at the dose of 1 μM.

These results show that the compounds according to the invention directly protect hepatocyte from cell death by inhibiting apoptosis.

Altogether, these results show that treatment with the compounds according to the invention reduce overt activation of the immune system via direct anti-inflammatory effects on macrophages in one hand, while they also directly reduce hepatocyte death on the other. Therefore, the compounds according to the invention display beneficial effects to treat patients with acute liver failure or acute-on-chronic liver failure.

1. A method for the treatment or prevention of liver failure in a subject in need thereof, comprising administering a pharmaceutically effective amount of a PPARα/γ agonist to the subject, wherein said PPARα/γ agonist is selected from the group consisting of aleglitazar, muraglitazar, tesaglitazar, a pharmaceutically acceptable salt thereof, and combinations thereof.

2. The method according to claim 1, wherein said PPARα/γ agonist is aleglitazar or a pharmaceutically acceptable salt thereof.

3. The method according to claim 1, wherein said PPARα/γ agonist is muraglitazar or a pharmaceutically acceptable salt thereof.

4. The method according to claim 1, wherein said PPARα/γ agonist is tesaglitazar or a pharmaceutically acceptable salt thereof.

5. The method according to claim 1, wherein the liver failure is selected from acute decompensation (AD), acute on chronic liver failure (ACLF), acute liver failure (ALF) and decompensated cirrhosis.

6. The method according to claim 1, wherein the subject has AD, decompensated cirrhosis with or without ACLF, or is at risk of AD and or ACLF.

7. The method according to claim 1, wherein the subject has decompensated cirrhosis, or is at risk of decompensated cirrhosis or acute decompensation.

8. The method according to claim 1, wherein the method is for the prevention of decompensated cirrhosis.

9. The method PPARα/γ agonist for use according to claim 1, wherein the method is for the reversion of decompensated cirrhosis to compensated cirrhosis.

10. The method PPARα/γ agonist for use according to claim 1, wherein the method is for the prevention of liver decompensation in the subject having ACLF.

11. The method according to claim 1, wherein the liver failure is ALF.

12. The method according to claim 1, wherein the method is for the prevention of kidney failure or for the prevention of hepatic encephalopathy.

13. The method according to claim 1, wherein the subject has ACLF without kidney failure, or wherein the subject has ACLF with a non-kidney organ failure with kidney dysfunction.

14. The method according to claim 1, in wherein the method is for the treatment of sepsis-associated ACLF.
